(19) United States
(12) Patent Application Publication
(10) Pub. No.: US 2002/0151018 A1
(43) Pub. Date: Oct. 17, 2002

(54) Stearoyl-CoA desaturase gene promoter

(76) Inventors: Stephen M. Prouty, Doylestown, PA (US); Lin Zhang, O'Fallon, MO (US); Kurt S. Stenn, Princeton, NJ (US)

Correspondence Address: AUDLEY A. CIAMPORCERO JR., JOHNSON & JOHNSON, ONE JOHNSON & JOHNSON PLAZA, NEW BRUNSWICK, NJ, 08933-7003, US

(21) Appl. No.: 10/016,725

(22) Filed: Oct. 30, 2001

Related U.S. Application Data
(60) Provisional application No. 60/244,508, filed on Oct. 31, 2000.

(51) Int. Cl. C12N 9/04; C07H 21/04; C12P 21/02; C12N 5/06

(52) U.S. Cl. 435/190; 435/69.1; 435/320.1; 435/325; 435/456; 536/23.2

(57) ABSTRACT

The present invention provides a promoter of the stearyl-CoA desaturase gene and functional moieties, fragments, and variants thereof, as well as nucleic acid constructs and vectors that contain such sequences, and the uses thereof.

### FIELD OF THE INVENTION

The present invention relates generally to gene promoters and the use thereof.

### BACKGROUND OF THE INVENTION

Stearoyl-CoA desaturase (Scd) is a lipid metabolic enzyme that catalyzes the insertion of a double bond between carbons 9 and 10 in the saturated fatty acids (SFA), palmitoyl-CoA and stearoyl-CoA, to generate the monounsaturated fatty acids (MUFA), palmitoleoyl-CoA and oleoyl-CoA, respectively. MUFA's play important roles in many processes including energy metabolism (triacylglycerol storage in adipose tissue) (Berry, 1997), membrane fluidity (Kates et al., 1984; Tiku et al., 1996), anti-oxidation (Parthasarathy et al, 1990; Diplock et al, 1988), signal transduction (Diaz-Guerra et al., 1991; Gyorfy et al, 1997; Kim et al, 1999), apoptosis (de Vries et al., 1997; Kasai et al, 1998), and senescence (Fukuchi-Mizutani et al, 1995; Kumar et al., 1999; Lee et al, 1999). Moreover, a critical role in differentiation is evidenced by the lack of development of the sebaceous and meibomian glands in the asebia mutant mouse, due to loss of SCD1 function (Zheng et al, 1999). Altered SCD gene expression is associated with various diseases such as cancer (Hrelia et al., 1994; Li et al, 1994), obesity (Jones et a., 1996), and aging (Kumar et al., 1999; Lee et al., 1999). The pleiotropic nature of MUFA's requires complex regulation of SCD that includes 1) hormones such as T3 (Waters et al., 1997a), insulin (Kasturi and Joshi, 1982; Prasad and Joshi, 1979; Waters and Ntambi, 1994) and estradiol (Lippiello et a., 1979), 2) dietary factors such as fasting (Oshino and Sato, 1972), carbohydrate (Prasad and Joshi, 1979; Waters and Ntambi, 1994; Jones et aL, 1998), vitamin A (Miller et al., 1997), polyunsaturated fatty acids (PUFA's) (Jeffcoat and James, 1978; Bossie and Martin, 1989; Sessler et al, 1996; Ntambi et al, 1996; Waters et al, 1997b; Ntambi, 1999), the metals iron (Rao et al., 1983) and copper (Wahle and Davies, 1975; Sreekrishna and Joshi, 1980), and peroxisomal proliferators, (Miller and Ntambi, 1996; Kurebayashi et a/., 1997), and 3) environmental factors such as temperature (Kasai et al., 1976; Tiku et al., 1996; Trueman et al, 2000). Furthermore, the rat Scd protein is shortlived (t½=3-4 hr) due to the action of an integral membrane-bound protease in the endoplasmic reticulum (Heinemann and Ozols, 1998).

In order to gain insight into the complex regulation of SCD genes, nucleotide sequences have been isolated from many organisms including plant, bacteria, protozoa, yeast, tick, fish, hamster, sheep, cow, rat, mouse, and human (Sakamoto et al, 1994; Ideta et al, 1995; Ward et al., 1997; Fukuchi-Mizutani et al, 1998; Tocher et al, 1998; Zheng et al., 1999). At the level of amino acid and nucleotide sequence, the SCD gene family exhibits both similarities and differences across the phylogenetic kingdom (Shanklin et al, 1994). The similarities are 1) the high levels of identity and similarity of the open reading frames (ORF's) among the various organisms, 2) the presence of conserved amino acids (and their spacing) involved in catalysis of the Δ9 desaturation reaction, and 3) the presence of a long 3′ untranslated region (UTR) in the vertebrate genes. The major difference is the variable number of SCD genes between different species. Those organisms with a single structural gene include cyanobacteria (Sakamoto et al, 1994), the yeast s. cerevisiae (Stukey et al., 1989), sheep (Ward et al., 1997), and human (Zhang et al, 1999) whereas those with more that one structural gene include mouse (Ntambi et al., 1988; Kaestner et al, 1989) and rat (Mihara, 1990), suggesting that SCD has undergone duplication and expansion within the rodent lineage. The reason for the multiple SCD genes in rodent is unclear, although there is recent evidence to suggest that mouse SCD1 and SCD2 have different substrate specificities (Kim and Ntambi, 1999). This hypothesis is supported by the finding of tissue specific expression of the rodent SCD isoforms (Ntambi et al, 1988; Kaestner et al., 1989; Mihara, 1990; Zheng et al, 1999), whereas the human (Zhang et al, 1999) and sheep (Ward et al, 1997) SCD are ubiquitously expressed. Further species-specific regulatory mechanisms are indicated by the use tandem polyadenylation signals by human SCD to generate 2 alternate mRNA transcripts (Zhang et al., 1999), each encoding the identical enzyme, whereas the rodent SCD isoforms generate a single mRNA transcript (Ntambi et al., 1988; Kaestner et al., 1989; Mihara, 1990).

Due to the diverse processes in which SCD functions, the regulation of SCD gene expression is involves both transcriptional and post-transcriptional mechanisms (Ntambi, 1995; Sessleret al, 1996; Sessler and Ntambi, 1998; Kim and Ntambi, 1999; Tabor et al, 1999). Studies of transcriptional regulation have focussed on functional analysis of promoters, these having been isolated from yeast s. cerevisciae OLE1(Stukey et al, 1990) and h. capsulatum OLE1(Tosco et al, 1997), mouse SCD1 (Ntambi et al., 1988) and SCD2 (Kaestner et al., 1989), rat SCD1 (Mihara, 1990), and sheep SCD (Ward et al., 1997). The mouse SCD1 promoter has been extensively analyzed and cis-elements identified that regulate transcription (Ntambi, 1995; Kim and Ntambi, 1999; Tabor et al., 1999).

Activation of mouse SCD1 transcription during 3T3-L1 adipocyte differentiation requires binding sites for 1) the differentiation-induced transcription factor SREBP (Tabor et al., 1999), and 2) the constitutive transcription factors NF-1 (Singh and Ntambi, 1998)and NF-Y (Tabor et al., 1999). The promoter of SCD2 contains the same cis elements and also requires the SREBP and NF-Y binding sites (Tabor et al., 1998; Tabor et al, 1999). Peroxisome proliferators have differential effects on SCD mRNA accumulation. The PPAR-γ ligand clofibrate activates transcription of mouse SCD1 via a PPRE located in the promoter (Miller and Ntambi, 1996), an effect suggested to account for maintenance of membrane fluidity during peroxisome proliferator induced expansion of cellular membranes. In contrast, the PPAR-γ ligand troglitazone suppresses accumulation of mouse SCD1 mRNA, an effect which may account for its use as an anti-obesity agent (Kurebayashi et al, 1997; Kim and Ntambi, 1999).

Due to the important role of unsaturated fatty acids (UFA's) in controlling fluidity of membranes and lipid stores, SCD gene expression is negatively regulated in vivo and in vitro by UFA's, both MUFA's (McDonough et al., 1992; Meesters et al., 1997) and PUFA's (Jeffcoat and James, 1978; Ntambi, 1995; Sessler and Ntambi, 1998; Ntambi, 1999). UFA-mediated repression of SCD mRNA has tissue-specific mechanisms, being transcriptional in liver (Sessler and Ntambi, 1998; Ntambi, 1999), and post-transcriptional in adipose tissue (Sessler et al., 1996). A functional PUFA response element has been identified in the mouse SCD1 (Waters et al., 1997b; Tabor et al, 1999) and SCD2 (Tabor et al., 1999) promoters that mediates repression in cultured hepatocytes (Waters et al., 1997b; Tabor et al., 1999) treated with a variety of PUFA's. This region encompasses the essential SREBP and NF-Y binding sites, suggesting that the PUFA-mediated repression involves destabilization of the cooperativity between SREBP and NF-Y (Tabor et al., 1999). Indeed, PUFA destabilize SREBP mRNA (Xu et al., 1999; Yahagi et al., 1999) suggesting that part of PUFA mediated supression of SCD mRNA is due to loss of the critical activator SREBP. In adipose tissue, on the other hand, PUFA induces the destabiliztion of SCD mRNA, possibly mediated by AUUUA destabilization sequences in the long 3′ UTR (Sessler et al., 1996), a common feature of all vertebrate SCD genes, including human.

In order to understand the role of SCD in human health and disease, we recently isolated the full length human SCD cDNA and structural gene, demonstrating the high homology to the mouse SCD genes, particularly with respect the ORF, intron-exon junctions, and the long 3′ UTR (Zhang et al, 1999). Unlike mouse and rat which have at least 3 SCD loci, human has only 2 loci, one being the structural gene and the other a fully processed pseudogene (Zhang et al., 1999). In this report, we characterize the human SCD promoter from the structural gene on chromosome 10. The human SCD promoter is similar to the mouse SCD promoters in that the PUFA response region is conserved and contains within it the critical SREBP and NF-Y binding sites required for mouse SCD1/SCD2 transcription. Further, we demonstrate that the CCAAT box (putative NF-Y binding site) binds nuclear proteins and is required for transcriptional activation in cultured keratinocytes. A minimal promoter, bearing this region, is responsive to UFA mediated transcriptional repression, as well as transcriptional activation by SFA. Finally, many putative transcription factor binding sites are identified that may play a role in regulating the diverse functions of SCD.

### SUMMARY OF THE INVENTION

The object of the present invention is to provide a promoter that causes high gene expression. Another object is to provide a promoter that can be used for selective transgenic expression into various tissues, such as the skin. The present invention provides a promoter of the stearyl-CoA desaturase gene, functional moieties of this gene, fragments, and variants thereof. Various embodiments provide, nucleic acid constructs and vectors that contain such sequences, their functional moieties, fragments, and variants, and the uses thereof.

In yet another embodiment the invention pertains to a pharmaceutical composition for treating a disease, said composition comprising a promoter of the stearoyl-CoA desaturase, a functional moiety, or a variant sequence thereof.

Other features and advantages of the present invention will be apparent from the detailed description of the invention and from the claims.

### BRIEF DESCRIPTION OF THE FIGURES

FIG. 1: Characterization of the 5′ end of the human SCD gene.

Sequence of the 5′ end of the human SCD gene. Numbering is relative to the translation initiation codon. Amino acids are indicated with single letter code. Start site of transcription in hair follicle tissue is boxed. Minor transcription sites common to hair follicles and HaCaT cells is dashed boxed. The FSE2 is double boxed. Termination of SCD cDNA clones 34 (filled arrowhead) and 45 (open arrowhead) are indicated. Arrow indicates the most 5′ extent of RNase-protected fragment in HaCaT keratinocyte cells. The core sequence of transcription factor binding sites, as determined by the Matinspector Professional search tool using the Transfac database, are underlined, with arrowheads indicating orientation. Restriction enzyme sites are indicated by caret marks above the sequence. Bold letters indicate the 5′ extent of the clones used for reporter assays.

FIG. 2 Comparison of the proximal promoter of mouse SCD1 and human SCD

Sequence alignment of the 5′ UTR and proximal promoter of mouse SCD1 (Ntambi et al., 1988) and human SCD. Numbering is relative to translation initiation site. Colon indicates identity between sequences. Transcription factor binding sites unique to human, as well as human sequence, are in italics. Transcription factor binding sites unique to mouse, as well as mouse sequence, are in regular font. Transcription factor binding sites common to mouse and human are bold. Arrowheads indicate orientation of transcription factor binding sites. Dashed box indicates FSE2. Solid boxes indicate transcription start site of mouse SCD1 in 3T3-L1 cells (Ntambi et al, 1988) and transcription start site of human SCD in human hair follicles.

FIG. 3: Determination of transcription initiation site

15 μg of total RNA from human hair follicles (lane 1) or HaCaT keratinocytes (lane 2) was hybridized with a 400 nt SCD riboprobe (302 nt is SCD sequence) at 42° C. After digestion of single-stranded RNA with RNase A/T1, the protected fragments were resolved along with a sequencing ladder of the probe-containing vector NPA2-pCR2.1. Filled arrowheads indicate major sites of transcription. Open arrowhead minor transcription start site common to hair follicles and HaCaT keratinocytes. Sizes (nt) of the protected fragments are indicated.

FIG. 4: Function analysis of human SCD Promoter

Upper Left: a panel of deletion constructs. Restriction sites are indicated. Sequence numbering is relative to translational start site. c1—is the reverse orientation of c1. Upper Right: promoter activity of constructs transfected into HaCaT cell line. Data is representative of at least two independent experiments. Constructs were transfected in duplicate, cell lysates harvested 48 h post-transfection, and readings performed in triplicate. Values are plotted as a ratio of the relative light units (RLU) of firefly luciferase to Renilla luciferase.

FIG. 5: Fine Deletion Analysis of the human SCD promoter

Left: panel of deletion constructs. Restriction sites and 5′ ends of primer sites are indicated. Right: promoter activity of constructs transfected into HaCaT cell line. Data is representative of at least two independent experiments. Constructs were transfected in duplicate, cell lysates harvasted 48 h post-transfection, and readings performed in triplicate. Values are plotted as a ratio of the relative light units (RLU) of firefly luciferase to Renilla luciferase. Lower panel: promoter region showing location of c9, c10, c6 (bold), location and orientation of transcription factor binding sites (underlined), nucleotides (boxed) subjected to site directed mutagenesis in FIG. 6, and oligonucleotides (lowercase) used for EMSA in FIG. 7.

FIG. 6: Site Directed Mutagenesis of SP-1 site and CCAAT box

Using c9 as a template, site directed mutagenesis of the SP-1 site (−551) and the CCAAT box (−501) were carried out as indicated in Example 6. Promoter activity of constructs transfected into HaCaT cell line is shown. Data is representative of at least two independent experiments. Constructs were transfected in duplicate, cell lysates harvasted 48 h post-transfection, and readings performed in triplicate. Values are plotted as a ratio of the relative light units (RLU) of firefly luciferase to Renilla luciferase. “Control” is transfection without plasmid DNA.

FIG. 7: EMSA of CCAAT box binding proteins

Nuclear proteins from HaCaT keratinoccytes were mixed with ³²P-labeled CCMT oligonucleotide, with and without competitor oligonucleotide, and analyzed using non-denaturing gel electrophoresis as described in Example 4. Sense strand of the double stranded oligonucleotides used for EMSA are indicated in red in FIG. 6; AP-2, −695 to −575; SP-1, −559 to −539; CCAAT, −509 to −489. Arrowhead indicates the band demonstrating specific CCAAT box binding proteins. The bar indicates bands that bind putative CCAAT box binding proteins with partial specificity.

FIG. 8: Comparison of the PUFA-response region of mouse and human SCD Genes

Sequence alignment of the PUFA-response region mouse SCD1 (Ntambi et al, 1988), mouse SCD2 (Kaestner et al., 1989), and human SCD. Numbering is relative to translation initiation site for all three genes. Colon indicates identity between sequences. All transcription factor binding sites and orientations (arrowheads) indicated are conserved among all three genes. SRE element is indicated in bold.

FIG. 9: SCD promoter activity in response to fatty acid treatment

24 hours following transfection of c9 into HaCaT keratinocytes, the cells were exposed to 100 uM of fatty acid/BSA for 24 hours. Cells were harvested and analyzed for luciferase activity. Data is representative of at least two independent experiments. Constructs were transfected in duplicate, cell lysates harvasted 48 h post-transfection, and readings performed in triplicate. Values are plotted as a ratio of the relative light units (RLU) of firefly luciferase to Renilla luciferase.

### DETAILED DESCRIPTION OF THE INVENTION

It is believed that one skilled in the art can, based upon the description herein, utilize the present invention to its fullest extent. The following specific embodiments are to be construed as merely illustrative, and not limitive of the remainder of the disclosure in any way whatsoever.

Unless defined otherwise, all technical and scientific terms used herein have the same meaning as commonly understood by one of ordinary skill in the art to which the invention belongs. Also, all publications, patent applications, patents, and other references mentioned herein are incorporated by reference.

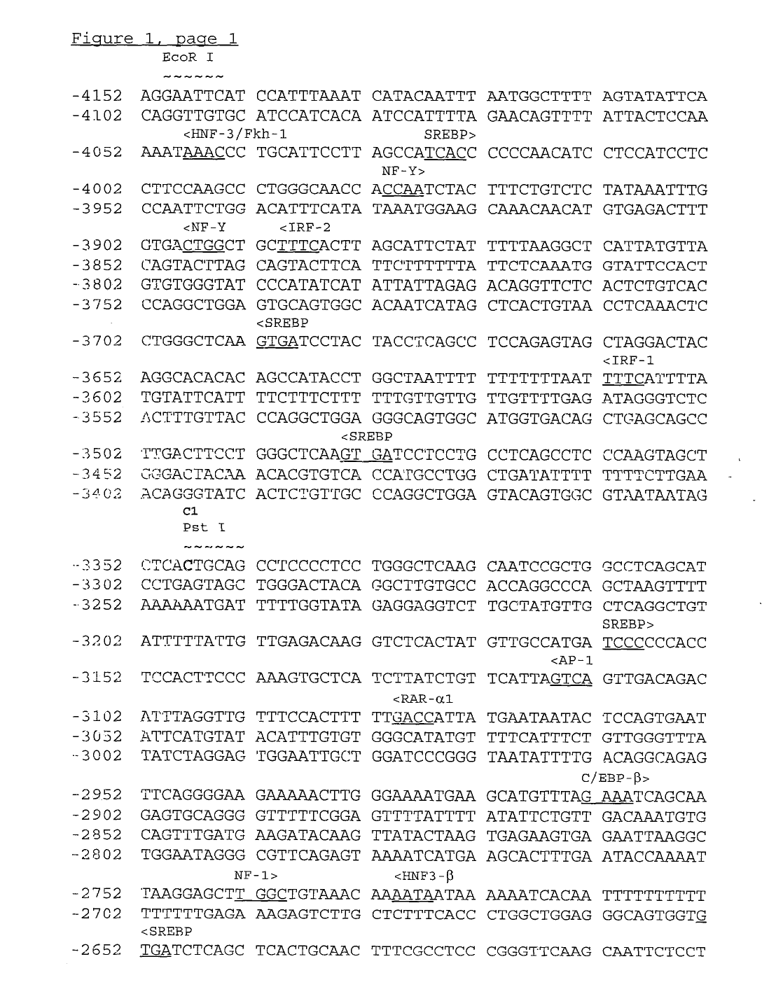

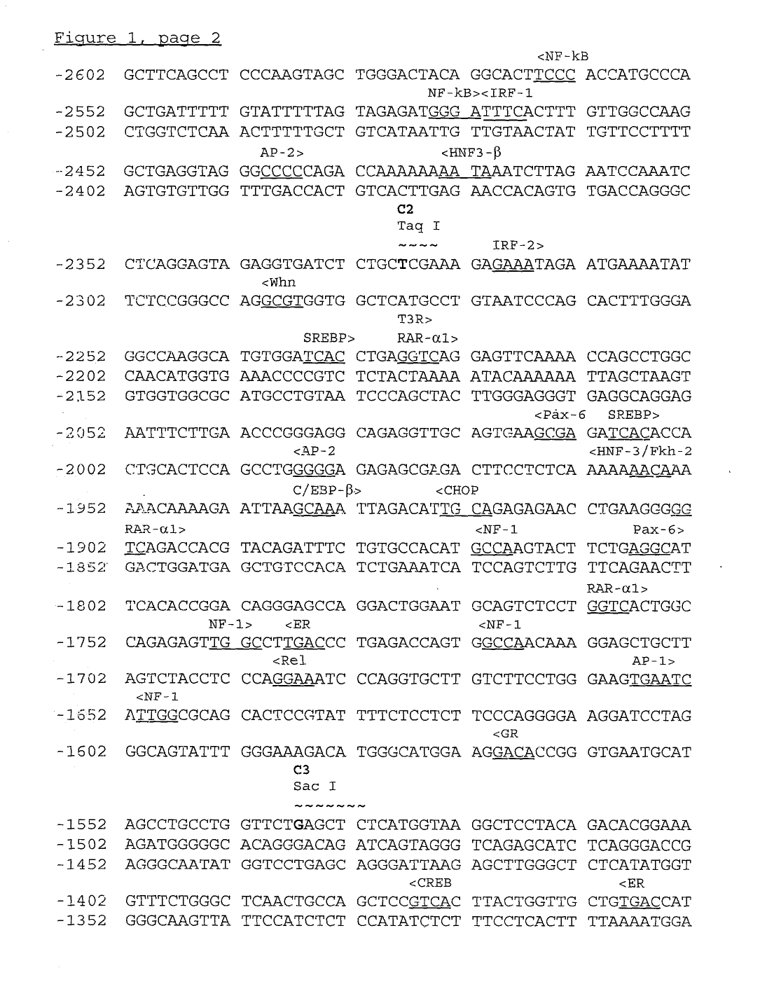

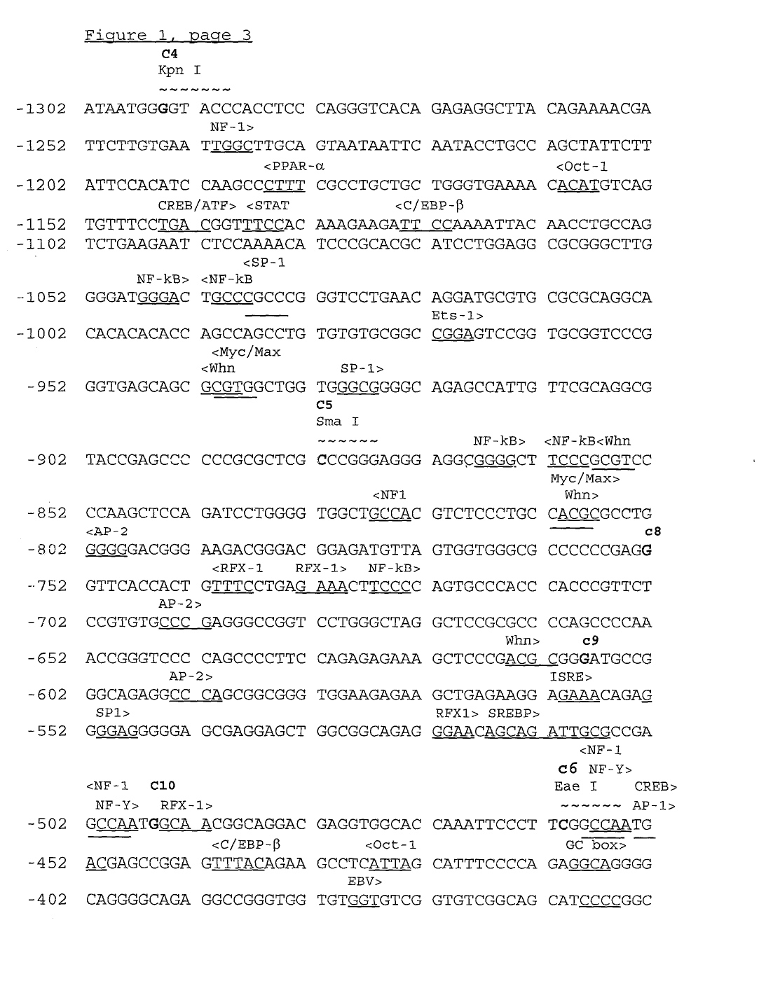

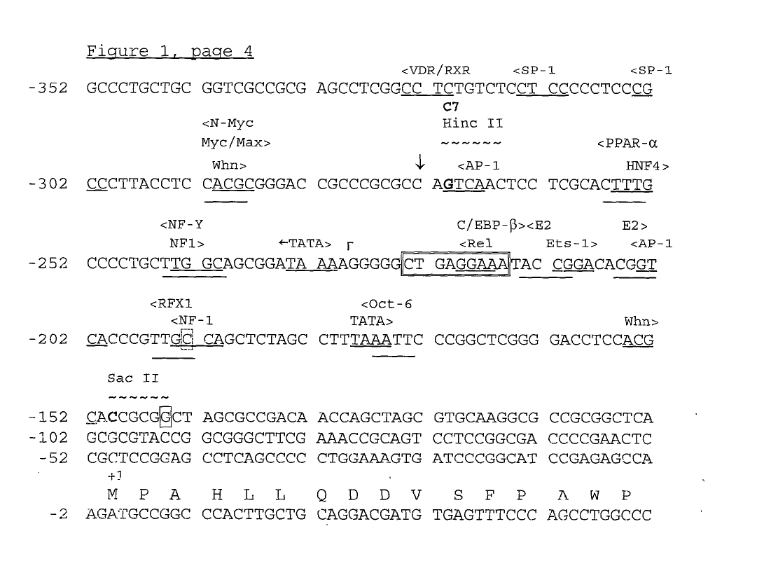

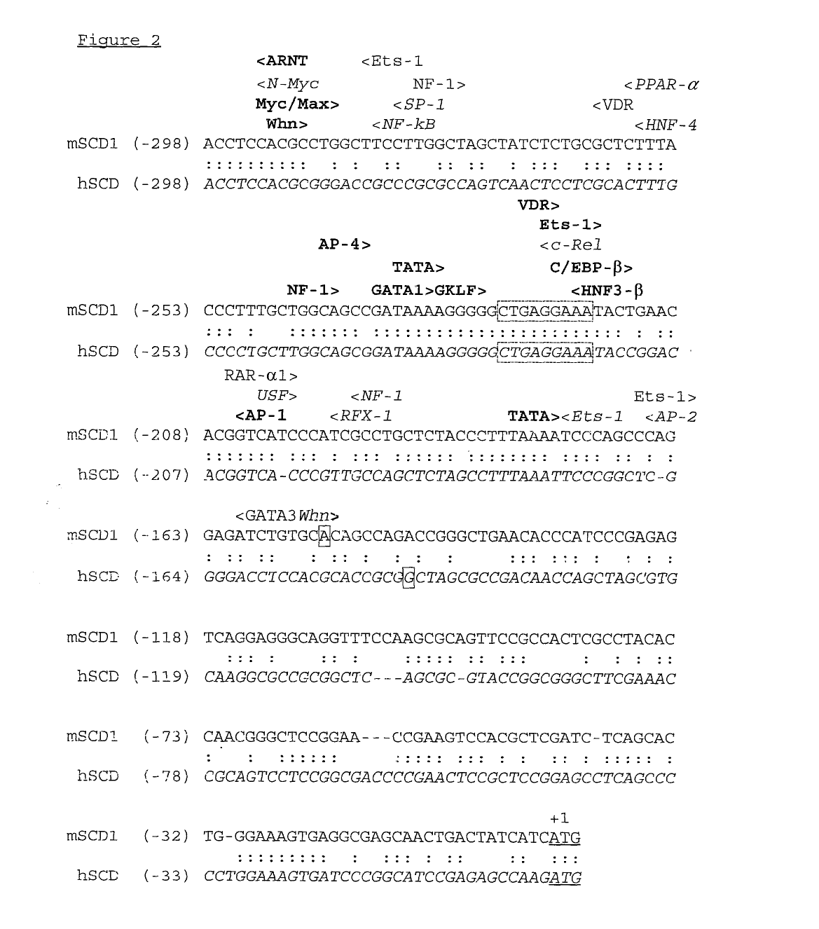

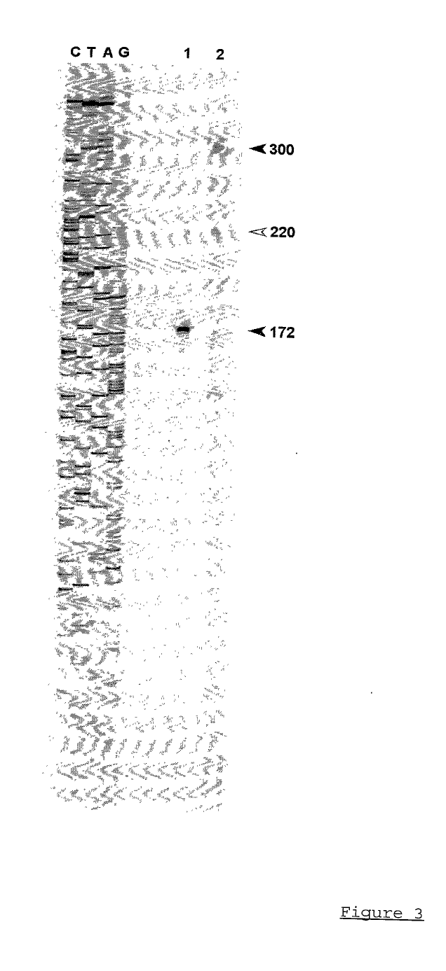

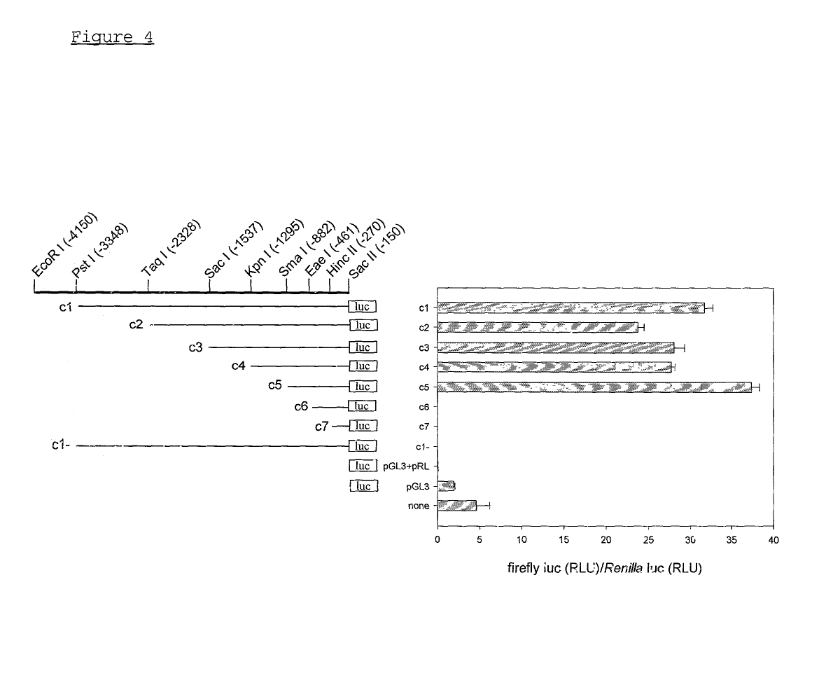

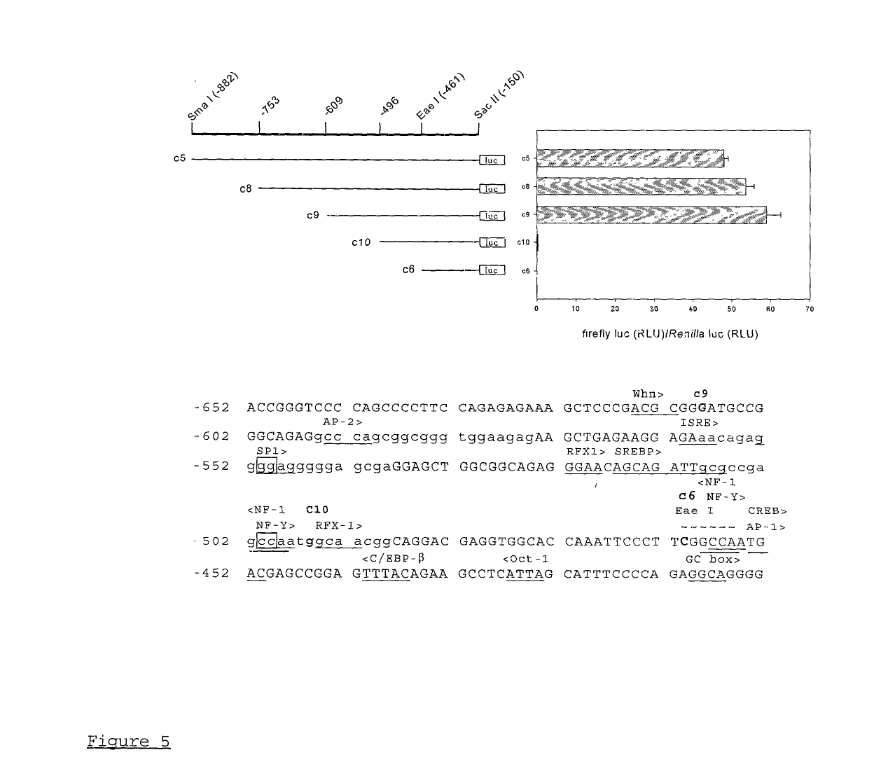

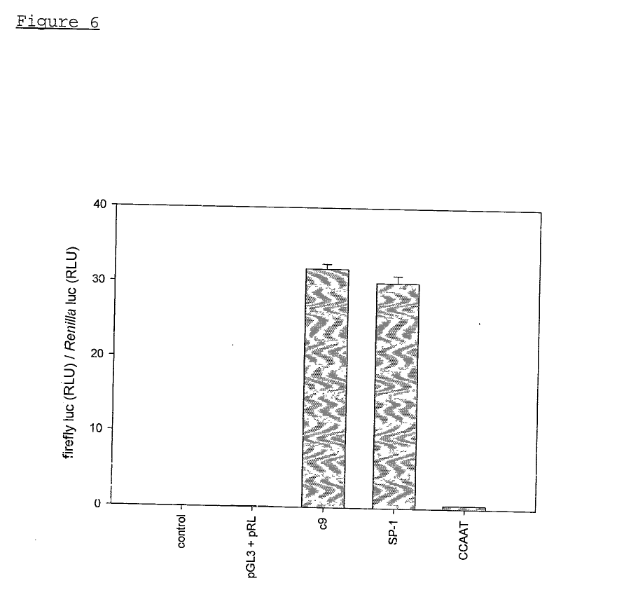

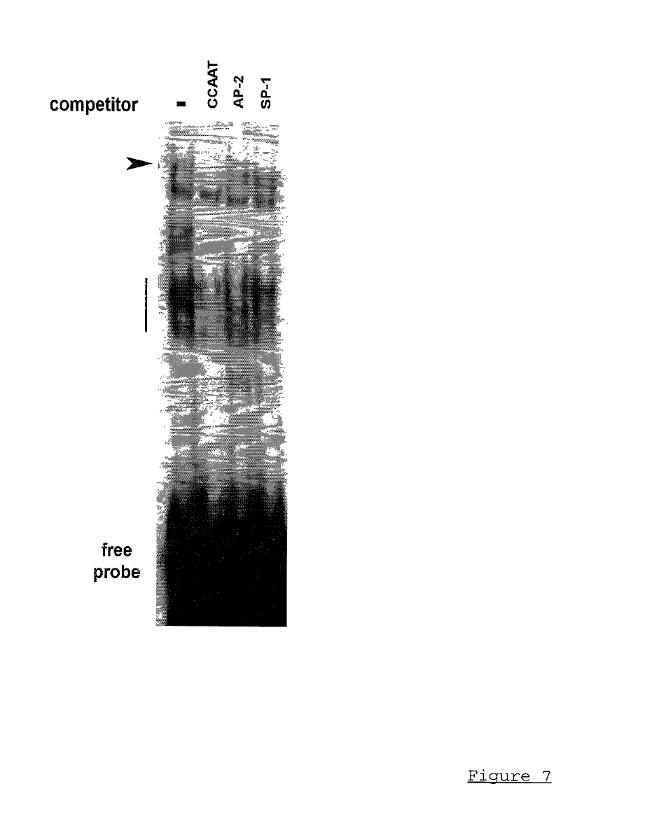

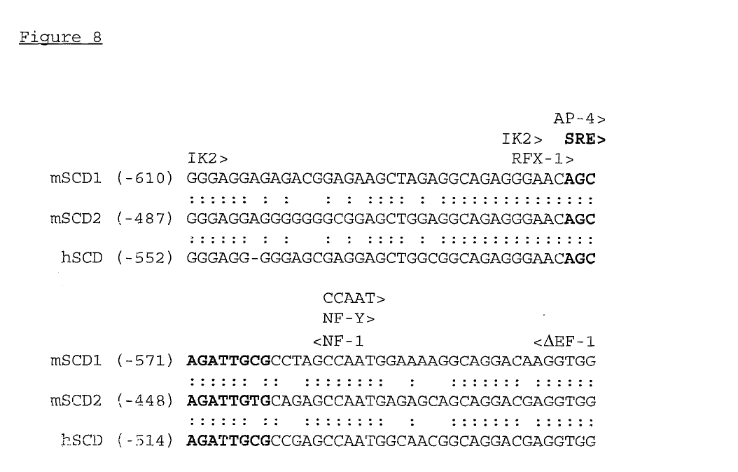

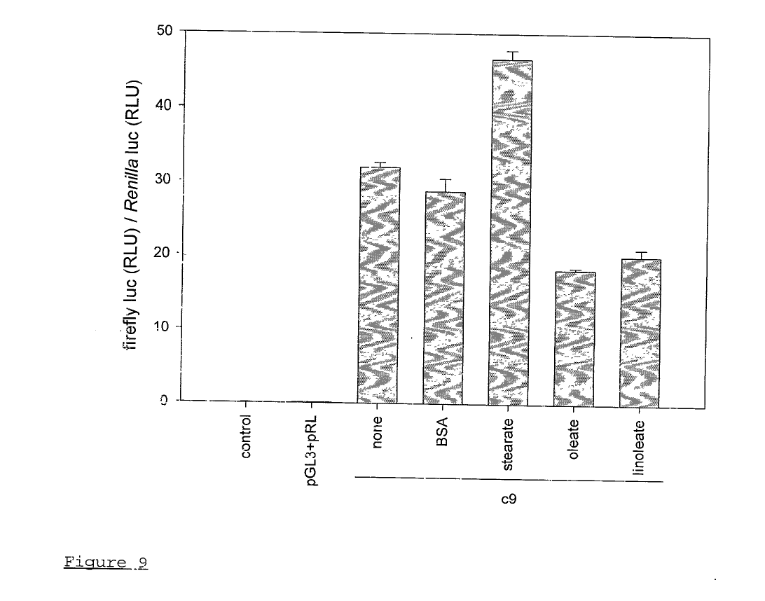

The present invention relates to a DNA whose nucleotide sequence comprising a sequence selected from the sequence from about 145 to about 4152 in the sequence listing in FIG. 1 which possesses promoter activity. The phrases “sequence selected from the base sequence from about 145 to about 4152 in the sequence listing in FIG. 1” and “sequence selected from the base sequence from about 193 to about 4152 in the sequence listing in FIG. 1” includes not only the entire sequences, but portions of these sequence that have promoter activity. This definition also includes functional moieties of the promoter. Thus, the present invention is not restricted to sequences selected from the base sequence from about 145 to about 4152 in the sequence listing in FIG. 1 (i.e. the entire sequence, and functional moieties thereof) but also comprises variants which possess promoter activity. Variants of this nature comprise, for example, deletions, additions, insertions and/or substitutions of one or more bases, preferably of from approximately 1 to approximately 50, in particular of from approximately 1 to approximately 25, especially of from approximately 1 to approximately 5, bases from a sequence selected from the base sequence from about 145 to about 4152 in the sequence listing in FIG. 1. Thus, a “variant” as termed here, is a modified sequence from the base sequence from about 145 to about 4152 in the sequence listing in FIG. 1 that still has promoter activity. Modifications can be made by various techniques that are known to the skilled artisan. An example of such a technique is given in Sambrook, J. et al., MOLECULAR CLONING. LABORATORY MANUAL, Second Edition, Cold Spring Harbor Laboratory Press (1989).

The promoter activity can be readily measured, for example, using the luciferase assay described herein and/or from the CAT or b-gal reporter assay methods described in Sambrook, J. et al., MOLECULAR CLONING. LABORATORY MANUAL, Second Edition, Cold Spring Harbor Laboratory Press (1989).

The present invention furthermore also relates to a nucleic acid construct which comprises a) at least one nucleic acid sequence of the promoter according to the invention (component a)) and, where appropriate, b) at least one effector gene (component b)), with the transcription of this effector gene being activated by component a). Component a) is preferably located upstream of component b).

A nucleic acid construct according to the invention is preferably composed of DNA. The term “nucleic acid construct” is understood as meaning artificial structures which are composed of nucleic acid and which can be transcribed in the target cells. They are preferably inserted into a vector, for example into non-viral vectors, such as plasmids, or viral vectors. The skilled artisan is familiar with the preparation of non-viral vectors and of viral vectors. Such preparation is explained, for example, in Sambrook, J. et al., MOLECULAR CLONING. LABORATORY MANUAL, Second Edition, Cold Spring Harbor Laboratory Press (1989).

The present invention also relates to host cells which harbor a nucleic acid construct according to the invention. Such host cells can be prokaryotic or eukaryotic, and advantageously are skin cells such as fibroblasts and keratinocytes.

The present invention also relates method for determining whether an agent effects (e.g., increases or decreases) the expression level of the effector gene (e.g., stearoyl-CoA desaturase such as human stearoyl-CoA desaturase) in a host cell of the present invention, which comprises the steps of (i) contacting the agent under suitable conditions with such a host cell expressing such effector gene at a known level; and (ii) determining whether the effector gene expression level increases or decreases after cellular contact with the agent, thereby determining whether the agent increases or decreases the expression level of the effector gene in skin cells already expressing the same. In one embodiment, the effector gene is a luciferase, β-galatosidase, or a cloramphenicol acetyltransferase gene.

The invention furthermore relates to a nucleic acid construct in which the promoter sequence according to the invention is combined with another target cell-specific, virus-specific, metabolism specific or cell cycle-specific promoter sequence and with at least one effector gene, in which this combination of promoter sequences controls the activation of at least one effector gene.

In general, the choice of an effector gene, in accordance with the invention, depends on the disease to be treated with the gene construct. Examples of effector genes are those used for the therapy of skin diseases (such as acne, psoriasis, and rosacea), tumor diseases, leukemias, autoimmune diseases, allergies, arthritis, inflammations, organ rejections, graft versus host reactions, diseases of the blood coagulation system, cardiovascular diseases, anemia, infections or damage to the CNS and are. described in detail in PCT Patent Applications WO 96/06940, WO 96/06938, WO 96/06941 and WO 96/06939.

A nucleic acid construct according to the invention can furthermore comprise two or more identical or different effector genes which are linked to each other by way of promoter sequences or internal ribosomal entry sites (IRES). Examples of these are given in the above mentioned patent applications. Methods for preparing these constructs are well known to the skilled artisan, as for example, described in the above mentioned patent applications which are incorporated by reference in their entireties.

A nucleic acid construct according to the invention can be used, for example, to express a gene 1) specifically in a skin cell, 2) specifically in a skin cell and also in a metabolism specific manner, 3) specifically in a skin cell and also cell cycle-specifically and 4) specifically in a skin cell and virus-specifically (and optionally cell cycle or metabolic specific). The controlled gene preferably encodes a pharmacologically active compound or an enzyme which cleaves an inactive precursor of a drug, thereby forming an active drug.

Preference is given to using a nucleic acid construct according to the invention to prepare a pharmaceutical composition (drug), for treating at least one of the above mentioned diseases, with the preparation of a pharmaceutical generally comprising the cloning of the nucleic acid construct into a suitable vector, which is then, for example, administered to the patient. A skilled artisan is familiar with other ways of using a promoter and these other ways are possible according to the invention. A skilled artisan also will appreciate creation and modification of various nucleic acid constructs according to the invention.

The pharmaceutical compositions contemplated are intended for parenteral, topical, oral or local administration and generally comprise a pharmaceutically acceptable carrier and an amount of the active ingredient sufficient to reverse or prevent the bad effects of a disease state. The carrier may be any of those conventionally used and is limited only by chemicophysical considerations, such as solubility and lack of reactivity with the compound, and by the route of administration.

Examples of pharmaceutically acceptable acid addition salts for use in the present inventive pharmaceutical composition include those derived from mineral acids, such as hydrochloric, hydrobromic, phosphoric, metaphosphoric, nitric and sulfuric acids, and organic acids, such as tartaric, acetic, citric, malic, lactic, fumaric, benzoic, glycolic, gluconic, succinic, p-toluenesulphonic acids, and arylsulphonic, for example.

The pharmaceutically acceptable carriers described herein, for example. vehicles, adjuvants, carriers or diluents, are well-known to those who are skilled in the art and are readily available to the public. It is preferred that the pharmaceutically acceptable carrier be one that is chemically inert to the active compounds and one that has no detrimental side effects or toxicity under the conditions of use.

The following examples are intended to describe the invention in more detail without limiting it.

### EXAMPLE 1

### Genomic Fragment Isolation

Isolation of the λ phage encompassing the 5′ end of the SCD gene (4 kb of promoter, exon 1, intron 1, exon 2, and part of intron 2) from a chromosome 10-specific genomic library was previously obtained using standard techniques (Zhang et al, 1999). The 5.6 kb insert was cloned as an EcoRI fragment into pBlueScript Stratagene, La Jolla, Calif., USA) (clone 19) and subsequently used to generate both nuclease protection assay probes and luciferase reporter assay constructs as described below.

### EXAMPLE 2

### Luciferase Reporter Construction

To generate p(−3348/−150)SCD-luc (c1), clone 19 was digested with PstI and SacII and blunted with T4 polymerase. All sequences herein are numbered relative to the translation start site, designated +1. The 3.2 kb fragment containing only the 5′ end of the gene was cloned into a blunted HindIII site of the luciferase vector pGL3-basic (Promega, Madison, Wis., USA). p(−2328/−150)SCD-luc (c2) and p(−1295/−150)SCD-luc (c4) were generated by digesting the clone 19 with TaqI/SacII and KpnI/SacII, respectively, and the resulting fragments were subcloned into a blunted HindIII site of pGL3-basic. p(−1537/−150)SCD-luc (c3) and p(−882/−150)SCD-luc (c5) were subsequently generated by digesting c1 with Sacl and SmaI, respectively. The resulting fragments were religated using T4 ligase. p(−461/−150)SCD-luc (c6) was generated by digesting c4 with EaeI/NcoI and the resulting fragment was ligated to blunted NcoI/HindIII sites of pGL3-basic. p(−270/−150)SCD-luc (c7) was created by digesting c4 with HincII and NcoI. The resulting fragment was subcloned into NcoI/HindIII digested, blunted, pGL-3 basic. p(−753/−150)SCD-luc (c8), p(−609/−150)SCD-luc (c9) and p(−496/−150)SCD-luc (c10) constructs were assembled by polymerase chain reaction using one of the three specific sense oligonucleotides (bp −753 to −732, c8), 5′-GGTTCACCACTGTTTCCTGAGA-3′; (−609 to −688, c9) 5′-GATGCCGGGCAGAGGCCCAGCG-3′;(−496 to −474, c10), 5′-GGCMCGGCAGGACGAGGTGGCA-3′; and a common antisense oligonucleotide (−166 to −145) 5′-CCGCGGTGCGTGGAGGTCCCCG-3′. All PCR reactions were conducted with proofreading Turbo PFU DNA polymerase (Stratagene, La Jolla, Calif., USA), and the original c5 construct was used as template. Amplification products were phosphorylated with T4 kinase and subcloned into the HindIII blunted site of pGL-3 basic. The sequence of the PCR products were examined for accuracy using the dideoxynucleotide chain termination method.

### EXAMPLE 3

### RNase Protection Analysis

Total RNA was extracted from human hair follicle biopsies and cultured HaCaT keratinocytes (Boukamp et al., 1988) using RNAzoI (TEL-TEST Inc., Friendswood, Tex., USA). A SCD fragment was amplified with PCR, using clone 19 as a template with the following primers: sense (−275 to −256), 5′GCCAGTCMCTCCTCGCACT3′; antisense (+7 to +27), 5′ATCGTCCTGCAGCAAGTGGGC3′. This resulted in a 302 bp fragment (nucleotide −275 to +27) which was subcloned into the TA cloning vector pCR2.1 (Invitrogen, Carlsbad, Calif., USA). The plasmid (NPA1-pCR2.1) was further digested by Xba1 and EcoRV to release unnecessary vector sequence between the insert and the T7 promoter and religated with T4 ligase. The final construct (NPA2-pCR2.1) was linearized by HindIII and a labeled antisense RNA (400 bp) was synthesized from the T7 promoter of the plasmid using T7 ANA polymerase (Ambion, Austin, Tex., USA). Following synthesis, the full-length probe was gel purified. Total RNA (15 ug per sample) from human hair follicles and HaCaT keratinocytes was hybridized to the labeled transcript for 16 h at 42° C. The RNA samples were digested with an RNase mixture (Ambion, Austin , Tex., USA), and the sizes of the protected RNA were determined by electrophoresis on a 5% denaturing polyacrylamide gel, using a DNA sequencing ladder generated from NPA2-pCR2.1.

### EXAMPLE 4

### Nuclear Extracts and Electrophretic Mobility Shift Assays

Nuclear extracts of HaCaT keratinocytes were prepared using standard procedures (Dignam et al., 1983). Protein concentrations of the extract were determined with BCA assay (Pierce, Rockford, Ill., USA). Electrophoretic mobility shift assays were performed with a Gel Shift assay system according to the manufacture's instruction (Promega, Madison, Wis., USA). Briefly, hybridization was performed at room temperature for 20 min in a volume of 20 ul consisting of 0.1 pmol of probe (70,000 cpm) and 5 ug of nuclear extract. The gel shift mixture was incubated for 20 min at room temperature, and samples were immediately electrophoresed at 150V for 3 h on a 5% nondenaturing acrylamide gel using 0.5×TBE running buffer (34 mM Tris.borate, 0.75 mM EDTA ) prerun at 100 V for 100 min. The gel was then dried for autoradiography. For competition studies, radioinert DNA competitor was added as a 100-fold molar excess and preincubated with the nuclear extract at room temperature for 5-10 min. The ³²P-labeled DNA was then added to the mixture and incubated at room temperature. The oligonucleotides used for EMSAs were as follows: AP-2 (sense, −595 to −575), 5′-GCCCAGCGGCGGGTGGAAGAG-3′; AP-2 (antisense), 5′-CTCTTCCACCCGCCGCTGGGC-3′; SP-1 (sense, −559 to −539), 5′-AACAGAGGGGAGGGGGAGCGA-3′; SP-1(antisense), 5′-TCGCTCCCCCTCCCCTCTGTT-3′; CCAAT (sense, −509 to −484), 5′GCGCCGAGCCAATGGCAACGG-3′; CAAT (antisense), 5′-CCGTTGCCATTGGCTCGGCGC-3′.

### EXAMPLE 5

### Transfection and Dual Luciferase Assay

The human keratinocyte cell line, HaCaT, was used as the recipient cells for transient transfection assays. The day prior to transfection, cells were seeded into 6 well plates at 400,000 cells per well. Transient transfections were performed with 2 ug of test plasmids using SuperFect Reagent (Qiagen, Valencia, Calif., USA) according to the manufacture's protocol. As a control for transfection efficiency and cell density, HaCaT cells were co-transfected with a 50 ng of Renilla luciferase control reporter vector driven by the thymidine kinase promoter (pRL-TK). 48 h post-transfection, adherent cells were washed once with 3 ml of phosphate-buffered saline, dissolved in 500 ul of cell culture lysis reagent (Promega, Madison, Wis., USA), and harvested after 30 mins at room temperature. Luciferase assays were performed immediately using a Dual-Luciferase Reporter Assay System kit (Promega, Madison, Wis., USA) and measurements were performed using TD-20/20 luminometer (Promega, Madison, Wis., USA) . The promoter activity was reflected by the ratio of firefly/Renilla luciferase for each construct. Triplicate readings were taken for each sample, all assays were performed in duplicate, and each experiment was repeated a minimum of two times.

### EXAMPLE 6

### Mutagenesis

Mutation in the binding sites for transcription factors SP-1 and CCAAT were introduced into the plasmid c9, containing the 0.5 kb SCD promoter subcloned into the blunted HindIII site of pGL-3 basic, using the Quick Change Site-Directed Mutagenesis kit (Stratagene, La Jolla, Calif., USA). Two double-stranded oligonuclotide primers containing the desired mutations are as follows (underlined base pairs denote mutant substitutions): 5′-AAGGAGAAACAGAGGAAAGGGGGAGCGAGGAGCTG-3′ (SP-1), and 5′-AGCAGATTGCGCCGAGAAAATGGCAACGGCAGGAC-3′(CCAAT).

### EXAMPLE 7

### Cell Culture

All reagents from Gibco BRL (Rockville, Md., USA) unless otherwise stated. HaCaT keratinocytes were routinely cultured in Dulbecco's modified Eagle's medium supplemented with 10% fetal calf serum (Sigma, St. Louis, Mo., USA), 2 mM L-glutamine, and 50 unit/ml penicillin/streptomycin. The cells were incubated in humidified incubators at 37° C. and 5% CO₂. For fatty acid treatments, HaCaT cells were exposed to fatty acid/BSA conjugate (100 uM) for 24 hours prior to harvest. Stock solutions (2.75 mM) of fatty acid/BSA solutions were prepared as follows: 8.5 mg stearate (Sigma: S3381) was dissolved in 1 ml 0.1 M NaOH then heated to 90° C. for dissolution. This solution was added dropwise to 6 ml of 10% fatty acid free BSA (Sigma: A7511) with stirring at 37° C. This solution was brought to 10 ml with dH2O, filtered through 0.22 uM filter, and stored at 4° C. The same procedure was followed for oleate (O3880), however heating was kept to a minimum. BSA solution used for controls was prepared following above procedure but without added fatty acids. Linoleate/BSA conjugate was purchased from Sigma (L8384) and reconstituted to 3 mM with dH2O. All stock solutions were diluted to 100 uM in cell culture media for application to cells.

### EXAMPLE 8

### DNA Sequencing

The DNA sequence in both directions was determined by Howard Hughes Medical Institute Biopolymer/Keck Foundation Biotechnology Resource Laboratory (Yale University, New Haven, Conn., USA) using Perkin Elmer Biosystems (Foster City, Calif., USA) model 337 gel DNA sequencer and model 3700 capillary DNA sequencer.

### EXAMPLE 9

### Characterization of the 5′-region of the SCD gene

Previously we have shown that there are two SCD loci in human genomre with one being the functional gene present on chromosome 10, and the other a pseudogene located on chromosome 17 (Zhang et al., 1999). To obtain the upstream sequence of the functional SCD gene, chromosome 10-specific libraries were screened with a 1.9 kb fragment (from clone 34) containing the 5′ end of the SCD cDNA (Zhang et al., 1999). One of the genomic clones obtained from this screen had a 5.6 kb insert (Zhang et al. 1999). Analysis of the insert indicated that there were approximately 4.0 kb upstream of the ATG translation start site.

Transcription factor binding sites were identified using the Matinspector Professional algorithm with the Transfac database v4.1 (Quandt et al., 1995) using core similarity setting of 0.75 and matrix similarity setting of 0.85. Many classes of transcription factor binding sites were identified, consistent with the pleiotropic function of SCD genes (FIG. 1). Two TATA boxes are present, being located at −179 and −234. Binding sites for constitutive transcription factors involved in basal promoter activity included Stimulating Protein 1 (SP-1) (−1038, −930, −551, −314, −304), CCAAT box binding proteins Nuclear Factor-1 (NF-1) (−2743, −1872, −1744, −1651, −1241, −827, −502, −459, −244) and Nuclear Factor-Y (NF-Y) (−3981, −3898, −501, −458), X box-binding protein Regulatory Factor X-1 (RFX-1) (−733, −522, −495, −196), Octamer binding proteins Oct-1 (−1160, −427) and Oct-6 (−177), AP-2 (−2440, −1987, −802, −695, −594, −194), Ets (−972, −212), cAMP Response Element Binding-Protein (CREB) (−1377, −1145, −454), and GC box-binding protein (−410). Although SCD is expressed in many tissues, its cellular pattern of expression within a tissue can be very specific, as seen in the mouse pilosebaceous unit (Zheng et al., 1999). Consistent with this is the presence of tissue-specific transcription factor binding sites such as skin-enriched Winged-helix nude (Whn) (−2290, −942, −858, −811, −615, −291, −155), liver-enriched Hepatocyte Nuclear Factor (HNF)-3β (−2730, −2424), HNF-3/Forkhead (Fkh) Homolog 1 (−4048), HNF-3/Fkh Homolog 2 (−1958), HNF-4 (−256), and eye-enriched Pax-6 (−2016, −1858). Desaturases are highly regulated in tissues engaged in lipid metabolism such as liver, skin, and adipose tissue. Accordingly, binding sites for transcription factors that regulate lipid metabolic genes are present in the SCD promoter, these being Sterol Regulatory Element Binding-Protein (SREBP) (−4027, −3692, −3484, −3612, −2236, −2010), Peroxisome Proliferator-Activated Receptor (PPAR)-γ (−1186, −257), and CCAAT Enhancer Binding-Protein (C/EBP)-β (−2913, −1946, −1124, −441). Moreover, a sequence similar to the Fat Specific Element 2 (FSE2), common to many lipid metabolic genes, was found at −224 nt. Consistent with the alteration of SCD mRNA levels by steroid hormones is the presence of binding sites for members cf the steroid hormone nuclear receptor superfamily including Vitamin D Receptor (VDR) (−324, −224; see FIG. X), Retinoic Acid Receptor (RAR)-α1 (−3080, −2228, −1904, −1762), Estrogen Receptor (ER) (−1738, −1359), Thyroid Hormone Receptor (T3R) (−2228), and Glucocorticoid Receptor (GR) (−1570). Consistent with a role in inflammation is the presence of Rel (−1689, −220), NF-κB (−2566, −2525, −1047, −1041, −868, −862, −727), Interferon Regulatory Factor (IRF)-1 (−3612, −2521), IRF-2 (−3890, −2320), Interferon Stimulated Response Element (ISRE) (−561), and Signal Transducers and Activators of Transcription (STAT) (−1138) binding sites. That SCD may play a role in viral infection is suggested by the presence of binding sites for papilloma virus E2 protein (−214, −206) and BRLF1 protein of Epstein-Barr Virus (EBV) (−379). Finally, a role for SCD in proliferation and differentiation is suggested by the presence of binding sites for the immediate early genes Myc/Max (−941, −291), Activator Protein (AP)-1 (−3116, −1678, −454, −271, −204), and N-Myc (−292).

A comparison of the 5′ UTR and proximal promoter of mouse SCD1 and human SCD reveal sequence identity of 64% between −298 to +1, suggestive of conserved regulatory elements (FIG. 2). The presence and position of the following elements is common to mouse SCD1 and human SCD promoters: TATA boxes, binding sites for Aryl Hydrocarbon Receptor (ARNT), Myc/Max, Whn, AP-4, GATA-1, Gut-Enriched Krueppel-like Factor (GKLF), and AP-1. Also common is the FSE2, which contains binding sites for VDR, Ets-1, HNF3-β, as well as a binding site for C/EBP-αan element in the mouse SCD1 promoter that binds C/EBP protein upon differentiation of 3T3-L1 cells (Christy et al, 1989) Furthermore, the NF-1 site (−245) that is required for mouse SCD1 transcription (Singh and Ntambi, 1998), is present in the human SCD promoter. The notable differences between the promoters in this region are the presence in the human of Rel, NF-κB, N-Myc, and RFX-1 binding sites.

### EXAMPLE 10

### Identification of the Transcription Initiation Site

Screening of a cDNA library from human foreskin keratinocytes for SCD resulted in 2 clones which extend 5′ from the ATG translation initiation, these being clone 34 to −235 and clone 45 to −229(Zhang et al, 1999) (arrowheads, FIG. 1). To further define the start site for transcription in skin cells, primer extension and Rnase protection were carried out with total RNA isolated from hair follicle biopsies and cultured HaCaT keratinocytes. Due to the high GC content in this region, primer extension did not yield consistent results. For the RNase protection assays, a cRNA probe, consisting of 302 nt of SCD (excluding vector sequence) from −275 to +27 was used in the protection assay. A major band of 172 nt was detected in total RNA isolated from hair follicles, whereas a major band of 300 nt was seen in HaCaT keratinocyte total RNA (FIG. 3). No bands of the size of full length probe (400 nt, including vector sequence) were detected (indicating lack of contaminating sense DNA template and in vitro transcribed sense RNA) nor was any smearing evident (indicating complete Rnase digestion). Liver showed the same protected fragments as hair follicles, whereas brain had a similar protection pattern to that of HaCaT cells (data not shown). This indicates that for hair follicles and liver, the major site of transcription initiation is at −145 (solid box in FIG. 1) from the translation start site. This position is 35 nt downstream of the proximal TATA box at −179 nt, similar to mouse SCD1 in 3T3-L1 cells (FIG. 2; ref (Ntambi et al., 1988)). In cultured keratinocytes, the transcription start site is 5′ to that of hair follicles, as indicated by the 300 nt protected fragment in HaCaT cell RNA and the termination of cDNA clones 34 and 45 in the region of the distal TATA box located at −234 nt (FIG. 3). There are several minor protected bands in HaCaT cells and skin, with a common band of 220 nt (FIG. 3). This indicates the presence of a minor transcription start site at −193, a position 42 nt downstream of the distal TATA box (single-lined, dashed box in FIG. 1). Brain RNA shows an identical protection pattern that of HaCaT cells (data not shown). Overall, these results suggest that the SCD gene has different major transcription start sites that depend on tissue specific factors.

### EXAMPLE 11

### Functional Analysis of the SCD Promoter

To determine if the sequences upstream of the transcription initiation site are sufficient to direct transcription, a 3.2 kb PstI-SacIl fragment of the 5′ flanking region was fused in both orientations to a promoterless luciferase reporter gene and examined for the ability to mediate basal transcription. In addition, progressive 3′ deletions in the putative promoter region were also examined for their effect on reporter activity. The resulting constructs were transiently transfected into the human keratinocyte cell line, HaCaT, and cell extracts were assayed for luciferase activity at 48 h post-transfection. A total 7 different constructs were initially generated using convenient restriction enzyme sites in the 3.2 kb flanking region (FIG. 4). Significant transcriptional activity was contained with 5 constructs covering the region between −3348 to −882 (FIG. 4). Further deletion to −461 resulted in complete loss of activity (FIG. 4). SCD sequences fused in opposite transcriptional orientation did not induce any significant level of reporter activity (FIG. 4). The highest promoter activity was obtained with construct c5 which corresponded to bp −882 to −150 (FIG. 4).

These results demonstrated that, of the constructs tested, c5 contained the minimal required nucleotide sequence information for SCD transcription, and that essential cis elements lay between −882 and −461.

To further delineate cis elements responsible for human SCD gene expression, the c5 construct was deleted further to create three more constructs, c8, c9, and c10 (FIG. 5). These construct were transiently transfected into HaCaT cells, in addition to the parental construct c5. Deletion of 130 nt from the 5′-end of the c5 construct (to generate c8) did not have any major effect on the promoter activity. Similarly, deletion of a further 144 nt, to generate c9, did not affect SCD promoter activity. However, deletion of a further 113 nt, to generate c10, completely removed the putative core promoter region as indicated by the reduced luciferase activity to background level (FIG. 5). Taken together, these results narrowed the region that contains essential cis elements required for SOD transcription in HaCaT keratinocytes to −609 to −498.

Examination of the sequences of this region revealed several potential transcription factor binding sites (FIG. 5, lower panel); AP-2 (−594), ISRE (−561), SP-1 (−551), RFX-1 (−522, −495), SREBP (−517), NF-1 (−502), and NF-Y (−501). Considering the role SP-1 and NF-Y in regulation of the Fatty Acid Synthase promoter (Roder et al., 1997), and NF-1 (Singh and Ntambi, 1998) and NF-Y (Tabor et al., 1999) in the regulation of mouse SCD genes, the effect of mutations in the SP-1 and CCMT box on SCD promoter activity was determined using site-directed mutagenesis. Using c9, the core binding site for SP-1 at −551 was changed from GGAG to AAAG (boxed in FIG. 5) and the CCMT box binding site for NF-1/NF-Y at −502/−501 was changed from CCM to AAAA (boxed in FIG. 5). Relative to the promoter activity of c9, mutation of the SP-1 site has no effect (FIG. 6). However, mutation of the CCMT box completely removes SCD promoter activity (FIG. 6). These results indicate that a critical cis element for SCD promoter activity is the CCAAT box located at −501.

### EXAMPLE 12

### EMSA for CCAAT Box Binding Proteins

To determine if the CCAAT box critical for SCD promoter activity was actually occupied by transcription factors present in HaCaT cell nuclei, we prepared nuclear extracts and performed EMSA (FIG. 7). A specific gel shift was obtained when nuclear extract from HaCaT cells was assayed with radiolabeled oligonucleotide corresponding to the CCAAT box binding site at −501 nt. The gel shifts were competed with non-radiolabeled CCAAT oligonucleotide, but not the AP-2 (−594) and SP-1 (−551) oligonucleotides. There are a few lower bands that were partially competed by CCAAT oligonucleotide but not by the AP-2 and Sp-1 oligonucleotides. Since the CCAAT oligonucleotide contains binding sites for 2 CCAAT box binding proteins, NF-1 and NF-Y, as well as a binding site for the X box binding protein, RFX-1, this suggests the possibility that the specific bands of different mobility are due to different complexes of these three transcription factors.

### EXAMPLE 13

### Regulation of the SCD Promoter by Unsaturated Fatty Acids

Previous work identified a region of the mouse SCD1 and SCD2 promoter that was responsible for down-regulation of promoter activity by UFA's (Waters et al., 1997b). This region of the human SCD promoter was compared to that of mouse SCD1 and SCD2 (FIG. 8). A striking degree of sequence similarity (99%) and identity (77%) between the three SCD genes is present in this region, suggesting conservation of important regulatory elements. It is notable that critical cis elements for mouse SCD1/SCD2 promoter activity, namely the CCAAT box element at −502/−501 (NF-1/NF-Y binding site) and the novel SRE at −517 are located within this region, and are conserved among all three genes. In addition, several other transcription factor binding sites are conserved among all three genes, these being IK2, AP-4, and RFX-1. To examine if the minimal SCD promoter (containing the UFA response region) could respond to UFA, c9 was transfected into HaCaT keratinocytes, and the cells subsequently exposed to various fatty acids conjugated to BSA (FIG. 9). BSA alone had no effect on SCD promoter activity. In contrast, stearate unregulated promoter activity, whereas oleate (a MUFA) and linoleate (a PUFA) down regulated promoter activity to the same extent. It is notable that stearate and oleate are the substrate and product, respectively, of the SCD enzyme. These results indicate that PUFA down-regulates transcription of human SCD, similar to mouse SCD1 and SCD2. Moreover, it shows that the substrate and product of the SCD enzyme can regulate transcription of the SCD promoter, through activation and repression, respectively.

References

1. Baldassarre, F., M. Mallardo, E. Mezza, G. Scala, and I. Quinto. 1995. Regulation of NF-kappa B through the nuclear processing of p105 (NF-kappa B1) in Epstein-Barr virus-immortalized B cell lines. J Biol Chem 270: 31244-8.

2. Berry, E. M. 1997. The biological properties of oleic acid. In Handbook of essential fatty acid biology: biochemistry, physiology, and behavioral neurobiology (ed. S. Yehuda and D. I. Mostofsky), pp. 89-101. Humana Press, Inc., Totowa, N.J.

3. Birkenmeier, E. H., P. C. Hoppe, K. A. Lyford, and B. Gwynn. 1992. Tissue- and cell-specific expression of human sn-glycerol-3-phosphate dehydrogenase in transgenic mice. Nucleic Acids Res 20: 2819-25.

4. Bossie, M. A. and Martin, C. E. Nutritional regulation of yeast delta-9 fatty acid desaturase activity. Journal of Bacteriology 171(12), 6409-6413. 89.

5. Boukamp, P., R. T. Petrussevska, D. Breitkreutz, J. Hornung, A. Markham, and N. E. Fusenig. 1988. Normal keratinization in a spontaneously immortalized aneuploid human keratinocyte cell line. J Cell Biol 106: 761-71.

6. Brissette, J. L., J. Li, J. Kamimura, D. Lee, and G. P. Dotto. 1996. The product of the mouse nude locus, Whn, regulates the balance between epithelial cell growth and differentiation. Genes Dev 10: 2212-21.

7. Brown, M. S. and J. L. Goldstein. 1997. The SREBP pathway: regulation of cholesterol metabolism by proteolysis of a membrane-bound transcription factor. Cell 89: 331-40.

8. Casimir, D. A. and Ntambi, J. M. cAMP activates the expression of stearoyl-CoA desaturase gene 1 during early preadipocyte differentiation. Journal of Biological Chemistry 271(47), 29847-29853. 96.

9. Choi, J. Y., J. Stukey, S. Y. Hwang, and C. E. Martin. 1996. Regulatory elements that control transcription activation and unsaturated fatty acid-mediated repression of the Saccharomyces cerevisiae OLE1 gene. J Biol Chem 271: 3581-9.

10. Christy, R. J., Yang, V. W., Ntambi, J. M., Geiman, D. E., Landschulz, W. H., Friedman, A. D., Nakabeppu, Y., Kelly, T. J., and Lane, M. D. Differentiation-induced gene expression in 3T3-L1 preadipocytes: CCAAT/enhancer binding protein interacts with and activates the promoters of two adipocyte-specific genes. Genes and Development 3(9), 1323-1335. 89.

11. Darlington, G. J., Ross, S. E., and MacDougald, O. A. The role of C/EBP genes in adipocyte differentiation. J Biol Chem 273(46), 30057-60. 98.

12. Darlington, G. J., N. Wang, and R. W. Hanson. 1995. C/EBP alpha: a critical regulator of genes governing integrative metabolic processes. Curr Opin Genet Dev 5: 565-70.

13. de Vries, J. E., Vork, M. M., Roemen, T. H., de Jong, Y. F., Cleutjens, J. P., van der Vusse, G. J., and van Bilsen, M. Saturated but not mono-unsaturated fatty acids induce apoptotic cell death in neonatal rat ventricular myocytes. J Lipid Res 38(7), 1384-1394. 97.

14. Diaz-Guerra, M. J., Junco, M., and Bosca, L. Oleic acid promotes changes in the subcellular distribution of protein kinase C in isolated hepatocytes. Journal of Biological Chemistry 266(35), 23568-23576. 91.

15. Dignam, J. D., R. M. Lebovitz, and R. G. Roeder. 1983. Accurate transcription initiation by RNA polymerase II in a soluble extract from isolated mammalian nuclei. Nucleic Acids Res 11: 1475-89.

16. Diplock, A. T., Balasubramanian, K. A., Manohar, M., Mathan, V. I., and Ashton, D. Purification and chemical characterization of the inhibitor of lipid peroxidation from intestinal mucosa. Biochim Biophys Acta 962(1), 42-50. 88.

17. Dooley, K. A., M. K. Bennett, and T. F. Osborne, 1999. A critical role for cAMP response element-binding protein (CREB) as a Co-activator in sterol-regulated transcription of 3-hydroxy-3-methylglutaryl coenzyme A synthase promoter. J Biol Chem 274: 5285-91.

18. Dooley, K. A., S. Millinder, and T. F. Osborne. 1998. Sterol regulation of 3-hydroxy-3-methylglutaryl-coenzyme A synthase gene through a direct interaction between sterol regulatory element binding protein and the trimeric CCAAT-binding factor/nuclear factor Y. J Biol Chem 273: 1349-56.

19. Enser, M. and J. L. Roberts. 1982. The regulation of hepatic stearoyl-coenzyme A desaturase in obese-hyperglycaemic (ob/ob) mice by food intake and the fatty acid composition of the diet. Biochem J 206: 561-70.

20. Ericsson, J., S. M. Jackson, J. B. Kim, B. M. Spiegelman, and P. A. Edwards. 1997. Identification of glycerol-3-phosphate acyltransferase as an adipocyte determination and differentiation factor 1- and sterol regulatory element-binding protein-responsive gene. J Biol Chem 272: 7298-305.

21. Fimia, G. M., D. De Cesare, and P. Sassone-Corsi. 1998. Mechanisms of activation by CREB and CREM: phosphorylation, CBP, and a novel coactivator, ACT. Cold Spring Harb Symp Quant Biol 63: 631-42.

22. Fukuchi-Mizutani, M., Savin, K., Cornish, E., Tanaka, Y., Ashikari, T., Kusumi, T., and Murata, N. Senescence-induced expression of a homologue of delta 9 desaturase in rose petals. Plant Mol Biol 29(4), 627-635. 95.

23. Fukuchi-Mizutani, M., Y. Tasaka, Y. Tanaka, T. Ashikari, T. Kusumi, and N. Murata. 1998. Characterization of delta 9 acyl-lipid desaturase homologues from Arabidopsis thaliana. Plant Cell Physiol 39: 247-53.

24. Graves, R. A., P. Tontonoz, S. R. Ross, and B. M. Spiegelman. 1991. Identification of a potent adipocyte-specific enhancer: involvement of an NF-1-like factor. Genes Dev 5: 428-37.

25. Gyorfy, Z., Benko, S., Kusz, E., Maresca, B., Vigh, L., and Duda, E. Highly increased TNF sensitivity of tumor cells expressing the yeast delta 9-desaturase gene. Biochem Biophys Res Commun 241(2), 465-470. 97.

26. Heinemann, F. S. and J. Ozols. 1998. Degradation of stearoylcoenzyme A desaturase: endoproteolytic cleavage by an integral membrane protease. Mol Biol Cell 9: 3445-53.

27. Herrera, R., H. S. Ro, G. S. Robinson, K. G. Xanthopoulos, and B. M. Spiegelman. 1989. A direct role for C/EBP and the AP-I-binding site in gene expression linked to adipocyte differentiation. Mol Cell Biol 9: 5331-9.

28. Horton, J. D., I. Shimomura, M. S. Brown, R. E. Hammer, J. L. Goldstein, and H. Shimano. 1998. Activation of cholesterol synthesis in preference to fatty acid synthesis in liver and adipose tissue of transgenic mice overproducing sterol regulatory element-binding protein-2. J Clin Invest 101: 2331-9.

29. Horvai, A. E., L. Xu, E. Korzus, G. Brard, D. Kalafus, T. M. Mullen, D. W. Rose, M. G. Rosenfeld, and C. K. Glass. 1997. Nuclear integration of JAK/STAT and Ras/AP-1 signaling by CBP and p300. Proc Natl Acad Sci USA 94: 1074-9.

30. Hrelia, S., Bordoni, A., Biagi, P. L., Galeotti, T., Palombini, G., and Masotti, L. The role of delta-6- and delta-9-desaturase in the fatty acid metabolism of hepatomas with different growth rate. Biochem Mol Biol Int 34(3), 449-455. 94.

31. Ideta, R., Seki, T., and Adachi, K. Sequence analysis and characterization of FAR-17c, an androgen-dependent gene in the flank organs of hamsters. Journal of Dermatological Science 9(2), 94-102. 95.

32. Inoue, J., R. Sato, and M. Maeda. 1998. Multiple DNA elements for sterol regulatory element-binding protein and NF-Y are responsible for sterol-regulated transcription of the genes for human 3-hydroxy-3-methylglutaryl coenzyme A synthase and squalene synthase. J Biochem (Tokyo) 123: 1191-8.

33. Jackson, S. M., J. Ericsson, R. Mantovani, and P. A. Edwards. 1998. Synergistic activation of transcription by nuclear factor Y and sterol regulatory element birding protein. J Lipid Res 39: 767-76.

34. Janknecht, R. and T. Hunter. 1996. Transcription. A growing coactivator network. Nature 383: 22-3.

35. Jeffcoat, R. and A. T. James. 1978. The control of stearoyl-CoA desaturase by dietary linoleic acid. FEBS Lett 85: 114-8.

36. Jones, B. H., M. A. Maher, W. J. Banz, M. B. Zemel, J. Whelan, P. J. Smith, and N. Moustaid. 1996. Adipose tissue stearoyl-CoA desaturase mRNA is increased by obesity and decreased by polyunsaturated fatty acids. Am J Physiol 271: E44-9.

37. Jones, B. H., M. K. Standridge, K. J. Claycombe, P. J. Smith, and N. Moustaid-Moussa. 1998. Glucose induces expression of stearoyl-CoA desaturase in 3T3-L1 adipocytes. Biochem J 335 ( Pt 2): 405-8.

38. Kaestner, K. H., Ntambi, J. M., Kelly, T. J. Jr, and Lane, M. D. Differentiation-induced gene expression in 3T3-L1 preadipocytes. A second differentially expressed gene encoding stearoyl-CoA desaturase. Journal of Biological Chemistry 264(25), 14755-14761. 89.

39. Kamei, Y., L. Xu, T. Heinzel, J. Torchia, R. Kurokawa, B. Gloss, S. C. Lin, R. A. Heyman, D. W. Rose, C. K. Glass, and M. G. Rosenfeld. 1996. A CBP integrator complex mediates transcriptional activation and AP-1 inhibition by nuclear receptors. Cell 85: 403-14.

40. Kasai, R., Kitajima, Y., Martin, C. E., Nozawa, Y., Skriver, L., and Thompson, G. A. Jr. Molecular control of membrane properties during temperature acclimation. Membrane fluidity regulation of fatty acid desaturase action? Biochemistry 15(24), 5228-5233. 76.

41. Kasai, T., K. Ohguchi, S. Nakashima, Y. Ito, T. Naganawa, N. Kondo, and Y. Nozawa. 1998. Increased activity of oleate-dependent type phospholipase D during actinomycin D-induced apoptosis in Jurkat T cells. J immunol 161: 6469-74.

42. Kasturi, R. and Joshi, V. C. Hormonal regulation of stearoyl coenzyme A desaturase activity and lipogenesis during adipose conversion of 3T3-L1 cells. Journal of Biological Chemistry 257(20), 12224-12230. 82.

43. Kates, M., E. L. Pugh, and G. Ferrante. 1984. Regulation of membrane fluidity by lipid desaturases. In Membrane fluidity (ed. M. Kates and L. A. Manson), pp. 379-395. Plenum Press, New York and London.

44. Kawa, K. 2000. Epstein-Barr virus--associated diseases in humans. Int J Hematol 71: 108-17.

45. Kim, J. H., Y. Kim, S. D. Lee, I. Lopez, R. S. Arnold, J. D. Lambeth, P. G. Suh, and S. H. Ryu. 1999. Selective activation of phospholipase D2 by unsaturated fatty acid. FEBS Lett 54: 42-6.

46. Kim, Y. C. and J. M. Ntambi. 1999. Regulation of stearoyl-CoA desaturase genes: role in cellular metabolism and preadipocyte differentiation. Biochem Biophys Res Commun 266: 1-4.

47. Kumar, V. B., K. Vyas, M. Buddhiraju, M. Alshaher, J. F. Flood, and J. E. Morley. 1999. Changes in membrane fatty acids and delta-9 desaturase in senescence accelerated (SAMP8) mouse hippocampus with aging. Life Sci 65: 1657-62.

48. Kurebayashi, S., T. Hirose, Y. Miyashita, S. Kasayama, and T. Kishimoto. 1997. Thiazolidinediones downregulate stearoyl-CoA desaturase 1 gene expression in 3T3-L1 adipocytes. Diabetes 46: 2115-8.

49. Kwok, R. P., J. R. Lundblad, J. C. Chrivia, J. P. Richards, H. P. Bachinger, R. G. Brennan, S. G. Roberts, M. R. Green, and R. H. Goodman. 1994. Nuclear protein CBP is a coactivator for the transcription factor CREB. Nature 370: 223-6.

50. Lee, C. K., R. G. Klopp, R. Weindruch, and T. A. Prolia. 1999. Gene expression profile of aging and its retardation by caloric restriction. Science 285: 1390-3.

51. Lee, K. N., M. W. Pariza, and J. M. Ntambi. 1996. Differential expression of hepatic stearoyi-CoA desaturase gene 1 in male and female mice. Biochim Biophys Acta 1304: 85-8.

52. Li, J., Ding, S. F., Habib, N. A., Fermor, B. F., Wood, C. B., and Gilmour, R. S. Partial characterization of a cDNA for human stearoyl-CoA desaturase and changes in its mRNA expression in some normal and malignant tissues. International Journal of Cancer 57(3), 348-352. 94.

53. Li, Y. C., A. E. Pirro, M. Amling, G. Delling, R. Baron, R. Bronson, and M. B. Demay. 1997. Targeted ablation of the vitamin D receptor: an animal model of vitamin D-dependent rickets type II with alopecia. Proc Natl Acad Sci USA 94: 9831-5.

54. Linhoff, M. W., K. L. Wright, and J. P. Ting. 1997. CCAAT-binding factor NF-Y and RFX are required for in vivo assembly of a nucleoprotein complex that spans 250 base pairs: the invariant chain promoter as a model. Mol Cell Biol 17: 4589-96.

55. Lippiello, P. M., Holloway, C. T., Garfield, S. A., and Holloway, P. W. The effects of estradiol on stearyl-CoA desaturase activity and microsomal membrane properties in rooster liver. Journal of Biological Chemistry 254(6), 2004-2009. 79.

56. Mach, B., V. Steimle, E. Martinez-Soria, and W. Reith. 1996. Regulation of MHC class II genes: lessons from a disease. Annu Rev Immunol 14:301-31.

57. Mantovani, R. 1999. The molecular biology of the CCAAT-binding factor NF-Y. Gene 239: 15-27.

58. McDonough, V. M., J. E. Stukey, and C. E. Martin. 1992. Specificity of unsaturated fatty acid-regulated expression of the Saccharomyces cerevisiae OLE1 gene. J Biol Cheni 267: 5931-6.

59. Meesters, P. A., J. Springer, and G. Eggink. 1997. Cloning and expression of the delta 9 fatty acid desaturase gene from Cryptococcus curvatus ATCC 20509 containing histidine boxes and a cytochrome b5 domain. Appl Microbiol Biotechnol 47: 663-7.

60. Mihara, K. Structure and regulation of rat liver microsomal stearoyl-CoA desaturase gene. Journal of Biochemistry 108(6), 1022-1029. 90.

61. Miller, C. W. and J. M. Ntambi. 1996. Peroxisome proliferators induce mouse liver stearoyl-CoA desaturase 1 gene expression. Proc Natl Acad Sci USA 93: 9443-8.

62. Miller, C. W., Waters, K. M., and Ntambi, J. M. Regulation of hepatic stearoyl-CoA desaturase gene 1 by vitamin A. Biochem Biophys Res Commun 231(1), 206-210. 97.

63. Natarajan, R., S. Ghosh, and W. M. Grogan. 1999. Regulation of the rat neutral cytosolic cholesteryl ester hydrolase promoter by hormones and sterols: a role for nuclear factor-Y in the sterol-mediated response. J Lipid Res 40: 2091-8.

64. Nehis, M., B. Kyewski, M. Messerle, R. Waldschutz, K. Schuddekopf, A. J. Smith, and T. Boehm. 1996. Two genetically separable steps in the differentiation of thymic epithelium. Science 272: 886-9.

65. Novina, C. D. and A. L. Roy. 1996. Core promoters and transcriptional control. Trends Genet 12: 351-5.

66. Ntambi, J. M. 1992. Dietary regulation of stearoyl-CoA desaturase 1 gene expression in mouse liver. J Biol Chem 267: 10925-30.

67. Ntambi, J. M. The regulation of stearoyl-CoA desaturase (SCD). Progress in Lipid Research 34(2), 139-150. 95.

68. Ntambi, J. M. 1999. Regulation ofstearoyl-CoA desaturase by polyunsaturated fatty acids and cholesterol. J Lipid Res 40: 1549-58.

69. Ntambi, J. M., Buhrow, S. A., Kaestner, K. H., Christy, R. J., Sibley, E., Kelly, T. J. Jr, and Lane, M. D. Differentiation-induced gene expression in 3T3-L1 preadipocytes. Characterization of a differentially expressed gene encoding stearoyl—CoA desaturase. Journal of Biological Chemistry 263(33), 17291-17300. 88.

70. Ntambi, J. M., A. M. Sessler, and T. Takova. 1996. A model cell line to study regulation of stearoyl-CoA desaturase gene 1 expression by insulin and polyunsaturated fatty acids. Biochem Biophys Res Commun 220: 990-5.

71. Oshino, N. and Sato, R. The dietary control of the microsomal stearyl CoA desaturation enzyme system in rat liver. Archives of Biochemistry and Biophysics 149(2), 369-377. 72.

72. Pahl, H. L. 1999. Activators and target genes of Rel/NF-kappaB transcription factors. Oncogene 18: 6853-6866.

73. Parthasarathy, S., J. C. Khoo, E. Miller, J. Barnett, J. L. Witztum, and D. Steinberg. 1990. Low density lipoprotein rich in oleic acid is protected against oxidative modification: implications for dietary prevention of atherosclerosis. Proc Natl Acad Sci USA 87: 3894-8.

74. Prasad, M. R. and V. C. Joshi. 1979. Regulation of rat hepatic stearoyl coenzyme A desaturase. The roles of insulin and carbohydrate. J Biol Chem 254: 997-9.

75. Quandt, K., Frech, K., Karas, H., Wingender, E., and Werner, T. Matlnd and Matinspector: new fast and versatile tools for detection of consensus matches in nucleotide sequence data. Nucleic Acids Res 23(23), 4878-84. 95.

76. Rao, G. A., R. T. Crane, and E. C. Larkin. 1983. Reduction of hepatic stearoyl-CoA desaturase activity in rats fed iron-deficient diets. Lipids 18: 573-5.

77. Roder, K., S. S. Wolf, K. F. Beck, and M. Schweizer, 1997. Cooperative binding of NF-Y and Sp1 at the DNase I-hypersensitive site, fatty acid synthase insulin-responsive element 1, located at −500 in the rat fatty acid synthase promoter. J Biol Chem 272: 21616-24.

78. Ross, S. R., R. A. Graves, A. Greenstein, K. A. Plaft, H. L. Shyu, B. Mellovitz, and B. M. Spiegelman. 1990. A fat-specific enhancer is the primary determinant of gene expression for adipocyte P2 in vivo. Proc Natl Acad Sci USA 87: 9590-4.

79. Sakamoto, T., H. Wada, I. Nishida, M. Ohmori, and N. Murata. 1994. delta 9 Acyl-lipid desaturases of cyanobacteria. Molecular cloning and substrate specificities in terms of fatty acids, sn-positions, and polar head groups. J Biol Chem 269: 25576-80.

80. Salbert, G., A. Fanjul, F. J. Piedrafita, X. P. Lu, S. J. Kim, P. Tran, and M. Pfahl. 1993. Retinoic acid receptors and retinoid X receptor-alpha down-regulate the transforming growth factor-beta 1 promoter by antagonizing AP-1 activity. Mol Endocrinol 7: 1347-56.

81. Segre, J. A., J. L. Nemhauser, B. A. Taylor, J. H. Nadeau, and E. S. Lander. 1995. Positional cloning of the nude locus: genetic, physical, and transcription maps of the region and mutations in the mouse and rat. Genomics 28: 549-59.

82. Sessler, A. M., N. Kaur, J. P. Palta, and J. M. Ntambi. 1996. Regulation of stearoyl-CoA desaturase 1 mRNA stability by polyunsaturated fatty acids in 3T3-L1 adipocytes. J Biol Chem 271: 29854-8.

83. Sessler, A. M. and J. M. Ntambi. 1998. Polyunsaturated fatty acid regulation of gene expression. J Nutr 128: 923-6.

84. Shanklin, J., Whittle, E., and Fox, B. G. Eight histidine residues are catalytically essential in a membrane-associated iron enzyme, stearoyl-CoA desaturase, and are conserved in alkane hydroxylase and xylene monooxygenase. Biochemistry 33(43), 12787-12794. 94.

85. Shimano, H., I. Shimomura, R. E. Hammer, J. Herz, J. L. Goldstein, M. S. Brown, and J. D. Horton. 1997. Elevated levels of SREBP-2 and cholesterol synthesis in livers of mice homozygous for a targeted disruption of the SREBP-1 gene. J Clin Invest 100: 2115-24.

86. Shimomura, I., H. Shimano, B. S. Korn, Y. Bashmakov, and J. D. Horton. 1998. Nuclear sterol regulatory element-binding proteins activate genes responsible for the entire program of unsaturated fatty acid biosynthesis in transgenic mouse liver. J Biol Chem 273: 35299-306.

87. Singh, M. V. and J. M. Ntambi. 1998. Nuclear factor 1 is essential for the expression of stearoyl-CoA desaturase 1 gene during preadipocyte differentiation. Biochim Biophys Acta 1398: 148-56.

88. Smith, S. B., H. J. Mersmann, E. O. Smith, and K. G. Britain. 1999. Stearoyl-coenzyme A desaturase gene expression during growth in adipose tissue from obese and crossbred pigs. J Anim Sci 77: 1710-6.

89. Sreekrishna, K. and V. C. Joshi. 1980. Inhibition of the microsomal stearoyl coenyme A desaturation by divalent copper and its chelates. Biochim Biophys Acta 619: 267-73.

90. Stone, R. L. and D. A. Bernlohr. 1990. The molecular basis for inhibition of adipose conversion of murine 3T3-L1 cells by retinoic acid. Differentiation 45: 119-27.

91. Stukey, J. E., V. M. McDonough, and C. E. Martin. 1989. Isolation and characterization of OLE1, a gene affecting fatty acid desaturation from Saccharomyces cerevisiae. J Biol Chem 264: 16537-44.

92. Stukey, J. E., McDonough, V. M., and Martin, C. E. The OLE1 gene of Saccharomyces cerevisiae encodes the delta 9 fatty acid desaturase and can be functionally replaced by the rat stearoyl-CoA desaturase gene. Journal of Biological Chemistry 265(33), 20144-20149. 90.

93. Swart, G. W., J. J. van Groningen, F. van Ruissen, M. Bergers, and J. Schalkwijk. 1997. Transcription factor C/EBP alpha: novel sites of expression and cloning of the human gene. Biol Chem 378: 373-9.

94. Swenson, J. J., A. E. Mauser, W. K. Kaufmann, and S. C. Kenney. 1999. The Epstein-Barr virus protein BRLF1 activates S phase entry through E2F1 induction. J Virol 73: 6540-50.

95. Tabor, D. E., J. B. Kim, B. M. Spiegelman, and P. A. Edwards. 1998. Transcriptional activation of the stearoyl-CoA desaturase 2 gene by sterol regulatory element-binding protein/adipocyte determination and differentiation factor 1. J Biol Chem 273: 22052-8.

96. Tabor, D. E., J. B. Kim, B. M. Spiegelman, and P. A. Edwards. 1999. Identification of conserved cis-elements and transcription factors required for sterol-regulated transcription of stearoyl-CoA desaturase 1 and 2. J Biol Chem 274: 20603-10.

97. Thewke, D. P., S. R. Panini, and M. Sinensky. 1998. Oleate potentiates oxysterol inhibition of transcription from sterol regulatory element1-regulated promoters and maturation of sterol regulatory element-binding proteins. J Biol Chem 273: 21402-7.

98. Tiku, P. E., Gracey, A. Y., Macartney, A. I., Beynon, R. J., and Cossins, A. R. Cold-induced expression of delta 9-desaturase in carp by Transcriptional and posttranslational mechanisms. Science 271(5250), 815-818. 96.

99. Tjian, R. and T. Maniatis. 1994. Transcriptional activation: a complex puzzle with few easy pieces. Cell 77: 5-8.

100. Tocher, D. R., M. J. Leaver, and P. A. Hodgson. 1998. Recent advances in the biochemistry and molecular biology of fatty acyl desaturases. Prog Lipici Res 37: 73-117.

101. Tosco, A., S. Gargano, G. S. Kobayashi, and B. Maresca. 1997. An AP1 element is involved in transcriptional regulation of delta9-desaturase gene of Histoplasma capsulatum. Biochem Biophys Res Commun 230: 457-61.

102. Trueman, R. J., P. E. Tiku, M. X. Caddick, and A. R. Cossins. 2000. Thermal thresholds of lipid restructuring and (&Dgr;)(9)-desaturase expression in the liver of carp (Cyprinus carpio L.). J Exp Biol 203 Pt 3: 641-50.

103. Wahle, E. W. and Davies, N. T. Effect of dietary copper deficiency in the rat on fatty acid composition of adipose tissue and desaturase activity of liver microsomes. British Journal of Nutrition 34(1), 105-112. 75.

104. Ward, R. J., Travers, M. T., Vernon, R. G., Salter, A. M., Buttery, P. J., and Barber, M. C. The ovine stearyl-CoA desaturase gene: cloning and determination of gene number within the ovine genome. Biochemical Society Transactions 25(4), S673. 97.

105. Waters, K. M., Miller, C. W., and Ntambi, J. M. Localization of a negative thyroid hormone-response region in hepatic stearoyl-CoA desaturase gene 1. Biochem Biophys Res Commun 233(3), 838-843. 97a.

106. Waters, K. M., C. W. Miller, and J. M. Ntambi. 1997b. Localization of a polyunsaturated fatty acid response region in stearoyl-CoA desaturase gene 1. Biochim Biophys Acta 1349: 33-42.

107. Waters, K. M. and Ntambi, J. M. Insulin and dietary fructose induce stearoyl-CoA desaturase. 1 gene expression of diabetic mice. Journal of Biological Chemistry 269(44), 27773-27777. 94.

108. Worgall, T. S., S. L. Sturley, T. Seo, T. F. Osborne, and R. J. Deckelbaum. 1998. Polyunsaturated fatty acids decrease expression of promoters with sterol regulatory elements by decreasing levels of mature sterol regulatory element-binding protein. J Biol Chem 273: 25537-40.

109. Xu, J., M. T. Nakamura, H. P. Cho, and S. D. Clarke. 1999. Sterol regulatory element binding protein-1 expression is suppressed by dietary polyunsaturated fatty acids. A mechanism for the coordinate suppression of lipogenic genes by polyunsaturated fats. J Biol Chem 274: 23577-83.

110. Yahagi, N., H. Shimano, A. H. Hasty, M. Amemiya-Kudo, H. Okazaki, Y. Tamura, Y. lizuka, F. Shionoiri, K. Ohashi, J. Osuga, K. Harada, T. Gotoda, R. Nagai, S. Ishibashi, and N. Yamada. 1999. A crucial role of sterol regulatory element-binding protein-1 in the regulation of lipogenic gene expression by polyunsaturated fatty acids. J Biol Chem 274: 35840-4.

111. Yang, C., L. H. Shapiro, M. Rivera, A. Kumar, and P. K. Brindle. 1998. A role for CREB binding protein and p300 transcriptional coactivators in Ets-1 transactivation functions. Mol Cell Biol 18: 2218-29.

112. Zacny, V. L., J. Wilson, and J. S. Pagano. 1998. The Epstein-Barr virus immediate-early gene product, BRLF1, interacts with the retinoblastoma protein during the viral lytic cycle. J Virol 72: 8043-51.

113. Zhang, J., Q. Y. Zhang, J. Guo, Y. Zhou, and X. Ding. 2000. Identification and Functional Characterization of a Conserved, Nuclear Factor 1-like Element in the Proximal Promoter Region of CYP1A2 Gene Specifically Expressed in the Liver and Olfactory Mucosa. J Biol Chem 275: 8895-8902.

114. Zhang, L., Ge, L., Parimoo, S., Stenn, K., and Prouty, S. M. Human stearoyl-CoA desaturase: alternative transcripts generated from a single gene by usage of tandem polyadenylation sites. Biochem J 340 ( Pt 1), 255-64. 99.

115. Zheng, Y., K. J. Eilertsen, L. Ge, L. Zhang, J. P. Sundberg, S. M. Prouty, K. S. Stenn, and S. Parimoo. 1999. Scd1 is expressed in sebaceous glands and is disrupted in the asebia mouse. Nat Genet 23: 268-70.

It is understood that while the invention has been described in conjunction with the detailed description thereof, that the foregoing description is intended to illustrate and not limit the scope of the invention, which is defined by the scope of the appended claims. Other aspects, advantages, and modifications are within the claims.

What is claimed is:

1. A DNA whose nucleotide sequence comprising the sequence selected from about 145 to about 4152 in the sequence listing in FIG. 1.

2. A DNA of claim 1, wherein said DNA comprises the sequence selected from about 193 to about 4152 in the sequence listing in FIG. 1.

3. A DNA of claim 1, wherein said DNA comprises the sequence from about 145 to about 4152 in the sequence listing in FIG. 1.

4. A DNA of claim 1, wherein said DNA comprises the sequence from about 193 to about 4152 in the sequence listing in FIG. 1.

5. A vector comprising a DNA of claim 1.

6. A vector of claim 5, wherein said vector is a plasmid or virus.

7. A nucleic acid construct comprising at least one DNA of claim 1.

8. A nucleic acid construct of claim 7, further comprising an effector gene, wherein said DNA activates the transcription of said effector gene.

9. A nucleic acid construct of claim 8, wherein said DNA is upstream of the effector gene.

10. A nucleic acid construct of claim 9, wherein said effector gene is a stearoyl-CoA desaturase gene.

11. A nucleic acid construct of claim 10, wherein said effector gene is human stearoyl-CoA desaturase gene.

12. A host cell that comprises a nucleic acid construct of claim 7.

13. A host cell that comprises a nucleic acid construct of claim 8.

14. A method for determining whether an agent effects the expression level of the effector gene in a host cell of claim 13, which comprises the steps of (i) contacting the agent under suitable conditions with such host cell expressing such effector gene at a known level; and (ii) determining whether the effector gene expression level increases or decreases after cellular contact with said agent.

15. A method of claim 14, wherein said effector gene is a stearoyl-CoA desaturase gene.

16. A method of claim 14, wherein said effector gene is human stearoyl-CoA desaturase gene.

17. A method of claim 14, wherein said effector gene is a luciferase gene.

18. A method of claim 14, wherein said effector gene is a β-galatosidase gene or a cloramphenicol acetyltransferase gene.

19. A composition comprising a nucleic acid that comprises the sequence from about 145 to about 4152 in the sequence listing in FIG. 1 having promoter activity, or a partial sequence of the promoter which possesses promoter activity, in a carrier.

20. A composition comprising a nucleic acid that comprises the sequence from about 193 to about 4152 in the sequence listing in FIG. 1 having promoter activity, or a partial sequence of the promoter which possesses promoter activity, in a carrier.
